(19) United States
(12) Patent Application Publication
(10) Pub. No.: US 2010/0063320 A1
(43) Pub. Date: Mar. 11, 2010

(54) METHOD FOR PRODUCING FORMIC ACID

(76) Inventors: Nina Challand, Mannheim (DE); Xavier Sava, Mannheim (DE); Michael Röper, Wachenheim (DE)

Correspondence Address: CONNOLLY BOVE LODGE & HUTZ, LLP, P O BOX 2207, WILMINGTON, DE, 19899, US

(21) Appl. No.: 12/532,642

(22) PCT Filed: Mar. 18, 2008
(86) PCT No.: PCT/EP08/53248

§ 371 Date: Sep. 23, 2009

(30) Foreign Application Priority Data
Mar. 23, 2007 (EP) 07104745.0

(51) Int. Cl. C07C 51/15

(52) U.S. Cl. 562/609

(57) ABSTRACT

The invention relates to a process for preparing formic acid, in which catalytic hydrogenation of carbon dioxide with hydrogen over a catalyst which comprises a metal of groups 8 to 10 of the Periodic Table in the presence of a primary, secondary and/or tertiary amine generates the corresponding ammonium formate and the ammonium formate is split by heating into formic acid and the amine, which comprises selecting the primary, secondary or tertiary amine from the amines of the formula I or mixtures thereof

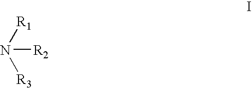

where R₁ to R₃ are the same or different and are each hydrogen, linear or branched alkyl radicals having from 1 to 18 carbon atoms, cycloaliphatic radicals having from 5 to 7 carbon atoms, aryl radicals and/or arylalkyl radicals, and at least one of the R₁ to R₃ radicals bears a hydroxyl group, and
performing the hydrogenation in a solvent which has a boiling point of 105° C. at standard pressure, and
obtaining the formic acid in the reaction mixture from the hydrogenation comprising the high-boiling solvent by thermally splitting the ammonium formate and distilling off the formic acid.

The present invention relates to a process for preparing formic acid.

It is known that ammonium formates of primary, secondary and/or tertiary amines can be obtained by catalytically hydrogenating carbon dioxide with hydrogen over hydrogenation catalysts in the presence of the primary, secondary and/or tertiary amines in a solvent. Formic acid can be released from the ammonium formates by heating.

Formic acid is prepared on the industrial scale in particular by carbonylation of methanol with carbon monoxide to give methyl formate and subsequent hydrolysis to formic acid with recovery of methanol (K. Weissermel, H.-J. Arpe, Industrielle organische Chemie [Industrial Organic Chemistry], fourth edition, VCH-Verlag, pages 45 to 46).

Instead of carbon monoxide, it is also possible to use carbon dioxide for the preparation of formic acid. The C₁ unit carbon dioxide is available in large amounts on earth in gaseous form or in bound form as carbonate.

It is known from numerous studies that carbon dioxide can be converted by electrochemical or photochemical reduction, but also by transition metal-catalyzed hydrogenation with hydrogen, to formic acid or its salts (W. Leitner, Angewandte Chemie 1995, 107, pages 2391 to 2405).

A method which appears promising on the industrial scale is in particular the catalytic hydrogenation of carbon dioxide in the presence of amines. The ammonium formates formed here can, for example, be cleaved thermally to formic acid and the amine used, which can be recycled into the hydrogenation.

EP 0 095 321 B1 (BP Chemicals) discloses the reaction of carbon dioxide with hydrogen in the presence of tertiary aliphatic, cycloaliphatic or aromatic amines, homogeneously dissolved compounds as catalysts which comprise metals of the eighth transition group, and lower alcohols or alcohol/water mixtures as solvents to corresponding ammonium formates. In Example 1, triethylamine, i-propanol/water mixtures and ruthenium trichloride are used. A disadvantage is the complicated workup of the hydrogenation effluent: first, the low boilers i-propanol (boiling point 82° C./1013 mbar), water and excess amine (boiling point of triethylamine 89.5° C./1013 mbar) have to be removed by distillation from the ammonium formates formed as high boilers.

To obtain the formic acid from the ammonium formates obtained after removal of the low boilers, they can be split thermally. The formic acid distilled off via the top (boiling point 100° C./1013 mbar) is, however, contaminated by the amine with a similar boiling point, which is partly distilled over with the formic acid, to reform the ammonium formate. Another problem is the removal and recycling of the homogeneous catalysts.

According to DE-A 44 31 233 too, Examples 1 to 4, carbon dioxide is hydrogenated in the presence of triethylamine, water and alcohols. The catalysts used are heterogeneous catalysts, for example ruthenium on Al₂O₃ as a support or ruthenium-comprising complexes on silicon dioxide as support. This mitigates the problem of catalyst recycling. However, the workup of the product mixture of the hydrogenation to obtain formic acid is afflicted with the same problems as in the process according to EP 0 095 321 B1.

EP 357243 B1 (BP Chemicals) discloses the hydrogenation of carbon dioxide in the presence of tertiary nitrogen bases such as triethylamine in a mixture of two different solvents which have a miscibility gap. In Example 1, for example, carbon dioxide is hydrogenated in the presence of triethylamine, ruthenium trichloride, tri-n-butylphosphine and the two solvents toluene and water. The hydrogenation effluent decomposes into a toluene phase which comprises the ruthenium catalyst, and an aqueous phase which comprises the triethylammonium formate formed. At page 3 line 56 to page 4 line 27, the nitrogen bases suitable for the inventive reaction are discussed. Mention is also made of primary, secondary or tertiary amines substituted by hydroxyl groups.

It is also known that ethanolamines can also be used in the hydrogenation of carbon dioxide in the presence of amines and [(m-C₆H₄SO₃⁻Na⁺)₃P]₃RhCl as a transition metal catalyst in aqueous solution (W. Leitner et al. in “Aqueous-Phase Organometallic Catalysis”, published by B. Cornils and W. A. Herrmann, Verlag WILEY-VCH, page 491ff.). However, the use of ethanolamines leads to significantly lower formate yields and lower TOF values than when triethylamine and dimethylamine are used. Within the ethanolamine series, formate yield and TOF value decrease starting from monoethanolamine through diethanolamine to triethanolamine (page 491, FIG. 2).

It is an object of the present invention to remedy the disadvantages mentioned and in particular to simplify the workup of the reaction effluents which occur in the catalytic hydrogenation of carbon dioxide in the presence of amines.

This object is achieved, surprisingly, by providing a process for preparing formic acid, in which catalytic hydrogenation of carbon dioxide with hydrogen over a catalyst which comprises a metal of groups 8 to 10 of the Periodic Table in the presence of a primary, secondary and/or tertiary amine generates the corresponding ammonium formate and the ammonium formate is split by heating into formic acid and the amine, which comprises selecting the primary, secondary or tertiary amine from the amines of the formula I or mixtures thereof

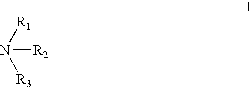

where R₁ to R₃ are the same or different and are each hydrogen, linear or branched alkyl radicals having from 1 to 18 carbon atoms, cycloaliphatic radicals having from 5 to 7 carbon atoms, aryl radicals and/or arylalkyl radicals, and at least one of the R₁ to R₃ radicals bears a hydroxyl group, and
performing the hydrogenation in a solvent which has a boiling point of ≧105° C. at standard pressure, and
obtaining the formic acid in the reaction mixture from the hydrogenation comprising the high-boiling solvent by thermally splitting the ammonium formate and distilling off the formic acid.

The inventive reaction can be illustrated, for example, in the case of use of triethanolamine as the tertiary base and [RuH₂(PPh₃)₄] as the hydrogenation catalyst, by the following reaction equation:

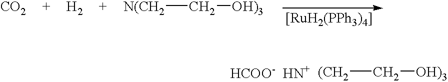

Carbon dioxide can be used in solid, liquid or gaseous form; it is preferably used in gaseous form.

In the amines of the formula I, the R₁ to R₃ radicals are the same or different and are each hydrogen, linear or branched alkyl radicals having from 1 to 18 carbon atoms, cycloaliphatic radicals having from 5 to 8 carbon atoms, aryl radicals having from 6 to 12 carbon atoms or arylalkyl radicals. At least one of the R₁ to R₃ radicals bears a hydroxyl group. The compounds of the formula I thus comprise one amino group and at least one hydroxyl group in the same molecule.

Useful linear alkyl radicals include, for example, methyl, ethyl, n-butyl, n-propyl, n-hexyl, n-decyl, n-dodecyl radicals.

Suitable branched alkyl radicals derive from linear alkyl radicals and bear, as side chains, alkyl radicals having from one to four carbon atoms, such as methyl, ethyl, propyl or butyl radicals. Preference is given to linear or branched alkyl radicals having not more than 14, more preferably not more than 10 carbon atoms.

Examples of useful cycloaliphatic radicals having from 5 to 8 carbon atoms include cyclopentyl or cyclohexyl radicals, which may be unsubstituted or substituted by methyl or ethyl radicals.

Useful aryl radicals include unsubstituted phenyl radicals or phenyl radicals which may be mono- or polysubstituted by C₁- to C₄-alkyl radicals.

Suitable aralkyl radicals are, for example, phenylalkyl radicals of the formula —CH₂—C₆H₅, whose phenyl group may be mono- or polysubstituted by C₁- to C₄-alkyl radicals.

At least one of the R₁ to R₃ radicals comprises a hydroxyl group. However, it is also possible that two or three of the R₁ to R₃ radicals comprise one hydroxyl group each. It may be a primary, secondary or tertiary hydroxyl group.

Preferably a total of two, more preferably three hydroxyl groups are present in the R₁ to R₃ radicals. As a result of the presence of hydroxyl groups, the R₁ to R₃ radicals become aliphatic or cycloaliphatic alcohols or become phenols.

Very particular preference is given to amines I with R₁ to R₃ radicals which are selected from the group consisting of C₁-C₁₄-alkyl, benzyl, phenyl and cyclohexyl, where the R₁ to R₃ radicals bear a total of from 1 to 3 hydroxyl groups.

Examples of the inventive amines I are ethanolamine, diethanolamine, triethanolamine, methyldiethanolamine, ethyldiethanolamine, dodecyldiethanolamine, phenyldiethanolamine, diphenylethanolamine, p-hydroxyphenyldiethanolamine, p-hydroxycyclohexylethylethanolamine, diethylethanolamine, dimethylethanolamine.

Tertiary amines I are preferred over primary and secondary amines I, for example the tertiary amines mentioned individually above. Very particular preference is given to triethanolamine.

Particularly preferred mixtures of amines I are mixtures of monoethanolamine, diethanolamine and triethanolamine, as obtained in the reaction of ethylene oxide with ammonia while varying the molar ratio (K. Weissermel, H.-J. Arpe, Industrielle Organische Chemie, fourth edition, VCH-Verlag, pages 172 to 173, 1994). These comprise, for example, from 10 to 75 mol % of monoethanolamine, from 20 to 25 mol % of diethanolamine and from 0 to 70 mol % of triethanolamine.

In general, the boiling point of the amines used in accordance with the invention at standard pressure (1013 mbar) is at least 130° C., preferably at least 150° C.

The hydrogenation catalyst comprises, as catalytically active components, one or more metals or compounds of these metals of groups 8 to 10 of the Periodic Table, i.e. the metals of the iron group, cobalt group and nickel group (Fe, Co, Ni, Ru, Rh, Pd, Os, Ir, Pt). Among these, preference is given to the noble metals (Ru, Rh, Pd, Os, Ir, Pt), very particular preference to palladium, rhodium and ruthenium. The catalytically active components comprise the metals themselves, but also compounds thereof, for example ruthenium trichloride and the complexes bis(triphenylphosphine)ruthenium dichloride and tris(triphenylphosphine)rhodium chloride. The metals mentioned and their compounds may be used in suspended or homogeneously dissolved form. However, it is also possible to apply the metals or their compounds to inert catalyst supports and to suspend the heterogeneous catalysts thus prepared in the inventive reaction or to use them in the form of fixed bed catalysts.

The inert catalyst supports used may, for example, be SiO₂, Al₂O₃, ZrO₂, mixtures of these oxides or graphite.

Particularly preferred catalysts are compounds of the formula RuH₂L₄ or RuH₂(LL)₂, in which L is a monodentate phosphorus-based ligand and LL is a bidentate phosphorus-based ligand.

When homogeneously dissolved metal compounds are used, the catalyst concentration is from 0.1 to 1000 ppm, preferable from 1 to 800 ppm, more preferably from 5 to 500 ppm of catalytically active metal, based on the overall reaction mixture.

A particularly preferred homogeneous catalyst is the ruthenium complex [RuH₂(triphenylphosphine)₄].

When heterogeneous catalysts are used, the amount of metal on the support is generally from 0.1 to 10% by weight of the heterogeneous catalyst.

The hydrogenation is performed in the presence of a high-boiling, generally organic solvent which, at standard pressure (1013 mbar) boils at a temperature at least 5° C., especially at least 10° C., higher than formic acid. Formic acid boils at from 100 to 101° C. at standard pressure. Examples of suitable solvents include alcohols, ethers, sulfolanes, dimethyl sulfoxide, open-chain or cyclic amides such as dialkylformamides, dialkylacetamides, N-formylmorpholine (boiling point 240° C./1013 mbar) or 5- to 7-membered lactams or mixtures of the compounds mentioned. In general, a homogeneous reaction mixture of high-boiling solvent and the amine(s) without a miscibility gap is present under the conditions of the hydrogenation.

The boiling point of the organic solvent used is preferably above 105° C., more preferably above 115° C.

Preferred solvents are, for example, dialkylformamides, dialkylacetamides and dialkyl sulfoxides, preferably having C₁-C₆-alkyl groups, and especially N,N-dibutylformamide (boiling point from 119 to 120° C., 15 mm), N,N-dibutylacetamide (boiling point from 77 to 78° C./6 mm of Hg) and dimethyl sulfoxide (boiling point 189° C.).

It is also possible to perform the inventive reaction without the addition of solvents which boil at a temperature higher than 105° C. at standard pressure and form only one liquid phase under the reaction conditions of the hydrogenation. In this case, the amines of the formula I themselves function as solvents.

The solvent mixture may comprise up to 5% by weight of water. Small amounts of water can, for example, be formed by esterification of alkanolamine and formic acid in the thermal splitting of the ammonium formates and the distillative formic acid removal.

The amount of solvent is from 5 to 80% by weight, especially from 10 to 60% by weight, based on the input mixture used.

The catalytic hydrogenation can be performed in the liquid phase in batchwise or preferably continuous mode.

The reaction temperature in the catalytic hydrogenation is generally from 30 to 150° C., preferably from 30 to 100° C., more preferably from 40 to 75° C.

The partial pressure of the carbon dioxide is generally from 5 bar up to 60 bar, especially from 30 bar up to 50 bar, the partial pressure of the hydrogen from 5 bar up to 250 bar, especially from 10 to 150 bar.

The molar ratio of carbon dioxide to hydrogen is generally from 10:1 to 0.1:1, preferably from 1:1 to 1:3.

The molar ratio of carbon dioxide to amine can be varied within the range from 10:1 to 0.1:1, preferably within the range from 0.5:1 to 2:1.

The residence time is generally from 10 minutes to 8 hours.

The process according to the invention features a higher solubility of carbon dioxide in the reaction mixture comprising the amines I: compare the solubility of CO₂ in triethylamine from: I. G. Podvigaylova at al. Sov. Chem. Ind. 5, 1970, pages 19 to 21 with the solubility of CO₂ in triethanolamine from: R. E. Meissner, U. Wagner, Oil and Gas Journal, Feb. 7, 1983, pages 55 to 58.

The ammonium formates prepared in accordance with the invention can be split thermally into formic acid and amine. According to the invention, this is done in the reaction mixture of the hydrogenation, which comprises the high-boiling solvent, if appropriate after preceding removal of the catalyst. The process according to the invention is notable in that distillative removal of the formic acid from the reaction mixture is readily possible, since formic acid is the component with the lowest boiling point. This allows it to be distilled easily out of the reaction mixture comprising the high-boiling solvent and the amine I.

To this end, the hydrogenation effluent is distilled in a distillation apparatus at pressures of from 0.01 to 2 bar, preferably from 0.02 to 1 bar, more preferably from 0.05 to 0.5 bar. This distils out the formic acid released via the top and condenses it. The bottom product, which consists of released amine I, solvent and if appropriate catalyst, is recycled into the hydrogenation stage. The bottom temperatures are, depending on the pressure set, from 130 to 220° C., preferably from 150 to 200° C.

Heterogeneous hydrogenation catalysts, which are used, for example, in suspension, are generally removed from the hydrogenation effluent by filtration before the thermal splitting of the formates. Depending on the thermal stability of the homogeneous hydrogenation catalysts, a removal before the thermal splitting of the ammonium formates may be advantageous, for example by extraction, adsorption or ultrafiltration.

For the thermal splitting, suitable apparatus is in particular distillation apparatus such as distillation columns, for example columns with structured packing, random packing and bubble-cap trays. Suitable random packings include, for example, preferably ceramic random packings to prevent corrosion. In addition, thin-film or falling-film evaporators may be advantageous when short residence times are desired.

In the formic acid removal, the mixture of high-boiling solvent and amine can be recycled into the carbon dioxide hydrogenation. Preference is given to a continuous process in which the solvent/amine mixture, if appropriate after removal of a purge stream, is circulated.

The invention is illustrated in detail by the examples which follow.

### EXAMPLES

### General Method for the Experiments on the Catalytic Hydrogenation of Carbon Dioxide with Hydrogen

In an autoclave, a mixture of an amine and a solvent in which [RuH₂(PPh₃)₄] catalyst had been dissolved was stirred intensively (600 revolutions per minute). At room temperature, hydrogen was then injected up to a pressure of 10 bar. The mixture was then heated to 50° C. and hydrogen was injected up to a pressure of 30 bar. Injecting carbon dioxide increased the pressure up to 60 bar. Subsequently, the mixture was stirred at 50° C. for one hour.

The autoclave was then cooled and decompressed. The formate content of the reaction effluent was determined by IC analysis. In Table 1, the feedstocks and their amounts are compiled together with the amounts of formate found and the turnover frequencies.

### Example 1 and Comparative Example 1

The experimental results show that, when triethanolamine in dibutylformamide is employed, TOFs in the same order of magnitude as when triethylamine in methanol is employed are achieved.

### Example 2 and Comparative Examples 2a and 2b

The three examples were performed with a quarter of the amount of catalyst from Example 1 and Comparative Example 1. They show that, when triethanolamine in dibutylformamide is employed, significantly better TOFs are achieved than when triethylamine in dibutylformamide or a dibutylformamide/water mixture is employed.

The results of Examples 1 and 2 are also surprising in that the carbon dioxide hydrogenation in water as a solvent with ethanolamines leads to significantly poorer results than with dimethyl- and triethylamine; cf. W. Leitner et al. in “Aqueous-Phase Organometallic Catalysis”, published by B. Cornils and W. A. Herrmann, Verlag WILEY-VCH, page 491.

TABLE 1

| Examples and | Catalyst | Amine | Solvent | Reaction | Formate formed | |
|---|---|---|---|---|---|---|
| Comparative Ex. | (mmol of Ru) | (g) (mmol) | (ml) | effluent (g) | (HCOO⁻) (% by wt.) | TOF ¹⁾ |
| Example 1 | [RuH₂(PPh₃)₄] | Triethanolamine | Dibutylformamide | 91.1 | 6.9 | 698 |
| | (0.2) | (60) | (40) | | | |
| | | (400) | | | | |
| Comparative | [RuH₂(PPh₃)₄] | Triethylamine | Methanol | 87.5 | 7.1 | 690 |
| Ex. 1 | (0.2) | (40) | (50) | | | |
| | | (400) | | | | |
| Example 2 | [RuH₂(PPh₃)₄] | Triethanolamine | Dibutylformamide | 88.6 | 0.42 | 165 |
| | (0.05) | (50) | (50) | | | |
| | | (335) | | | | |
| Comparative | | Triethylamine | Dibutylformamide | 90.2 | 0.26 | 104 |
| Ex. 2a | | (5.1) | (100) | | | |
| | | (50) | | | | |
| Comparative | | | Dibutylformamide | 84.0 | 0.12 | 45 |
| Ex. 2b | | | (100) | | | |
| | | | 1% by wt. of water | | | |

¹⁾ TOF (turnover frequency) = moles of target product per mole of catalyst metal and hour; TON (turnover number); TOF = TON per hour; all experiments were performed for one hour

1.-12. (canceled)

13. A process for preparing formic acid in which catalytic hydrogenation of carbon dioxide with hydrogen over a catalyst which comprises a metal of groups 8 to 10 of the Periodic Table in the presence of at least a primary, secondary or tertiary amine or a mixture thereof generates the corresponding ammonium formate and splitting the ammonium formate by heating into formic acid and the amine, which comprises selecting the primary, secondary or tertiary amine from the amines of the formula I or mixtures thereof 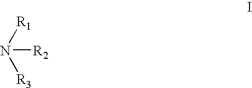
where R₁ to R₃ are the same or different and are each hydrogen, linear or branched alkyl radicals having from 1 to 18 carbon atoms, cycloaliphatic radicals having from 5 to 7 carbon atoms, aryl radicals and/or arylalkyl radicals, and at least one of the R₁ to R₃ radicals bears a hydroxyl group, and
performing the hydrogenation in a solvent which has a boiling point of >105° C. at standard pressure, and
obtaining the formic acid in the reaction mixture from the hydrogenation comprising the high-boiling solvent by thermally splitting the ammonium formate and distilling off the formic acid.

14. The process according to claim 13, wherein the amine is monoethanolamine, diethanolamine or triethanolamine or a mixture of two or three of these compounds.

15. The process according to claim 13, wherein the catalyst comprises ruthenium, rhodium or palladium or a mixture thereof.

16. The process according to claim 13, wherein the catalyst is a homogeneous catalyst or a suspended or fixed-bed heterogeneous catalyst.

17. The process according to claim 13, wherein the catalyst comprises a compound of
the formula RuH₂L₄ or RuH₂(LL)₂, in which L is a monodentate phosphorus-comprising ligand and LL is a bidentate phosphorus-comprising ligand.

18. The process according to claim 13, wherein the catalyst comprises the compound [RuH₂(PPh₃)₄], in which case the compound may be present in the reaction mixture in homogenously dissolved form or in heterogeneous form on a support.

19. The process according to claim 13, wherein the high-boiling solvent is an alcohol, ether, sulfolane, sulfoxide, open-chain or cyclic amide or mixtures thereof.

20. The process according to claim 19, wherein the solvent is selected from the group consisting of N,N-dialkylformamides, N,N-dialkylacetamides, N-formylmorpholine, 5- to 7-membered N-alkyllactams and dialkyl sulfoxides where alkyl is in each case C₁- to C₅-alkyl, and mixtures thereof.

21. The process according to claim 19, wherein the high-boiling solvent is N,N-dibutylformamide or dimethyl sulfoxide.

22. The process according to claim 13, wherein the catalytic hydrogenation is performed at temperatures of from 30° C. to 150° C.

23. The process according to claim 13, wherein the amine I and high-boiling solvent form a monophasic mixture under the conditions of the hydrogenation.

24. The process according to claim 13, wherein the mixture of the amine I and high-boiling solvent is recycled into the hydrogenation after the formic acid has been distilled off.
